(19) United States
(12) Patent Application Publication
(10) Pub. No.: US 2012/0283604 A1
(43) Pub. Date: Nov. 8, 2012

(54) ULTRASOUND NEUROMODULATION TREATMENT OF MOVEMENT DISORDERS, INCLUDING MOTOR TREMOR, TOURETTE'S SYNDROME, AND EPILEPSY

(76) Inventor: David J. Mishelevich, Playa del Rey, CA (US)

(21) Appl. No.: 13/467,009

(22) Filed: May 8, 2012

Related U.S. Application Data
(60) Provisional application No. 61/483,734, filed on May 8, 2011.
(60) Provisional application No. 61/538,936, filed on Sep. 25, 2011.
(60) Provisional application No. 61/542,288, filed on Oct. 3, 2011.

(51) Int. Cl. A61N 7/00

(52) U.S. Cl. 601/2

(57) ABSTRACT

Disclosed are methods and systems and methods for non-invasive neuromodulation using ultrasound to treat movement disorders (e.g., tremor disorders such as Parkinson's Disease and essential tremor, Tourette's Syndrome, and epilepsy). Also included are the Tourette's vocalizations. The neuromodulation can produce acute or long-term effects. The latter occur through Long-Term Depression (LTD) and Long-Term Potentiation (LTP) via training Included is control of direction of the energy emission, intensity, frequency, pulse duration, firing pattern, and phase/intensity relationships to targeting and accomplishing up regulation and/or down regulation.

### CROSS REFERENCE TO RELATED APPLICATIONS

This patent application claims priority to Provisional Patent Application Nos. 61/483,734 filed May 8, 2011, entitled “ULTRASOUND NEUROMODULATION TREATMENT OF MOTOR DISORDERS,” 61/538,936, filed Sep. 25, 2011, entitled “ULTRASOUND NEUROMODULATION TREATMENT OF TOURETTE'S SYNDROME,” and 61/542,288, filed Oct. 3, 2011, entitled “ULTRASOUND NEUROMODULATION TREATMENT OF EPILEPSY.”

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity for the treatment of a medical condition.

### BACKGROUND OF THE INVENTION

It has been demonstrated that focused ultrasound directed at neural structures can stimulate those structures. If neural activity is increased or excited, the neural structure is up regulated; if neural activated is decreased or inhibited, the neural structure is down regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996;22 (2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3 (10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, William, James P., PCT/US2009/050560, WO 2010/009141, published Jan. 21, 2011).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. In cases different than treatment of motor disorders, the stimulation frequency for inhibition is 300 Hz or lower (depending on condition and patient). In the case of motor disorders, based on experience with Deep-Brain Stimulation (DBS) with implanted electrodes, treatment for Parkinson's Disease or Essential Tremor would typically be 130 pulse per second and Dystonia in the range of 135-185 pulses per second (all superimposed on the carrier frequency of say 0.5 MHz or similar) although this will be both patient and condition specific. Repeated sessions result in long-term effects. In other situations, high frequencies (defined as being in the range of 500 Hz to 5 MHz) are excitatory and activate neural circuits. This works whether the target is gray or white matter. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. Patent Application 12/263,026 published as US 2009/0112133 A1, Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and FA Jolesz, “Demonstration of potential noninvasive ultrasound brain therapy through an intact skull,” Ultrasound Med Biol, 1998 February; 24 (2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

Because of the utility of ultrasound in the neuromodulation of deep-brain structures, it would be both logical and desirable to apply it to the treatment of motor disorders.

### SUMMARY OF THE INVENTION

It is the purpose of this invention to provide methods and systems for non-invasive neuromodulation using ultrasound to treat movement disorders (e.g., motor tremor disorders such as Parkinson's Disease and essential tremor, Tourette's Syndrome, and epilepsy). Also included are the Tourette's vocalizations. Such neuromodulation can produce acute effects or Long-Term Potentiation (LTP) or Long-Term Depression (LTD). Included is control of direction of the energy emission, intensity, frequency, pulse duration, and phase/intensity relationships to targeting and accomplishing up-regulation and/or down-regulation. Use of ancillary monitoring or imaging to provide feedback is optional. In embodiments where concurrent imaging is performed, the device of the invention is constructed of non-ferrous material.

MOTOR TREMOR DISORDERS: Targets useful for the treatment of motor disorders have been demonstrated with the results of Deep Brain Stimulation (DBS) therapy. Typically DBS is used when the quality of life of the patient decreases to an unsatisfactory level on medications of the side effects of those medications become severe. Areas that control movement are the subthalamic nucleus (STN), the ventralis intermedius nucleus of the thalamus (Vint), and the internal globus pallidus (GPi). For Parkinson's disease (PD), those structures are the subthalamic nucleus (STN) or globus pallidus interna (GPi).

In the case of motor disorders, based on experience with Deep-Brain Stimulation (DBS) with implanted electrodes, treatment for Parkinson's Disease or Essential Tremor would typically be 130 pulse per second and Dystonia in the range of 135-185 pulses per second (all superimposed on the carrier frequency of say 0.5 MHz or similar and may be divided into pulses 0.1 to 20 msec. repeated at intervals of 2 Hz or shorter) although this will be both patient and condition specific. For example in difficult cases it may be that rates up to 250 Hz or down to 50 Hz may be more effective.

For essential tremor (ET), the structure is the ventro intermediate nucleus of the thalamus (Vint), and for dystonia, the GPi or STN is stimulated. Unilateral DBS is used for essential tremor (e.g., for suppression of upper-extremity tremor) and bilateral DBS is used for PD and dystonia.

In terms of outcomes, 50% improvement in motor outcomes, rigidity, tremor, or dyskinesias. For essential tremor there has been 80-90%. For generalized dystonia, the improvement is 50-70% and up to 50% for secondary dystonia such as spasmodic torticollis). Medtronic DBS Therapy for Dystonia is indicated as an aid in the management of chronic, intractable (drug refractory) primary dystonia, including generalized and segmental dystonia, hemidystonia, and cervical dystonia (torticollis).

As to contraindications, Dementia is a contraindication for DBS treatment, but need not be so for ultrasound neuromodulation. Other DBS contradictions include exposure to MRI using a full-body RF coil or a head transmit coil that extends over the chest area, diathermy, and other devices such as cardiac pacemakers, cardioverter/defibrillators, external defibrillators, ultrasonic equipment, electrocautery, or radiation therapy. Again, these need not be contraindications for ultrasound neuromodulation.

TOURETTE'S SYNDROME: Multiple targets can be neuromodulated singly or in groups to treat Tourette's Syndrome, whether motor tics or vocalizations. To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS). Targets for treating Tourette's Syndrome have been identified such as the hippocampus and amygdala (Peterson, BS, Choi, HA, Hoa, X, Amat, JA, Zhu, H, Whiteman, R, Liu, J, Xu, D, and R Bansal, “Morphologic features of the amygdala and hippocampus in children and adults with Tourette syndrome,” Arch Gen Psychiatry. 2007 November; 64 (11):1281-91), both of which would be down regulated.

Other potential targets are the thalamus, sub-thalamic nuclei, and basal ganglia. Targets depend on specific patients and relationships among the targets. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

EPILEPSY: Multiple targets can be neuromodulated singly or in groups to treat epilepsy. To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS). Targets for treating epilepsy have been identified such as the Hippocampus, Temporal Lobe, Thalamus, and the Cerebellum. Most targets are identified through evaluating the effect of Deep-Brain Stimulation (DBS)(Boon P, Raedt R, de Herdt V, Wyckhuys T, and K Vonck, “Electrical stimulation for the treatment of epilepsy,” Neurotherapeutics. 2009 April; 6 (2):218-27.

Other potential targets are the Amygdala, Dentate Nucleus, and Mamillary Body. Targets depend on specific patients and relationships among the targets. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging. Tyler (PCT/US2009/050560, WO 2010/009141) lists motor disorders as a potential area of treatment, but does not pass the test of embodiment because neither the relevant targets nor approaches are described.

While ultrasound can be focused down to a diameter on the order of one to a few millimeters (depending on the frequency), whether such a tight focus is required depends on the conformation of the neural target.

### BRIEF DESCRIPTION OF THE DRAWINGS

FIG. 1 shows a block diagram of the control circuit.

FIG. 2 shows a block diagram of the feedback circuit for neuromodulation mitigation of motor-control disorders.

FIG. 3 shows unilateral ultrasonic-transducer targeting of the ventro intermediate nucleus for the treatment of Essential Tremor.

FIG. 4 shows bilateral ultrasonic-transducer targeting of the Subthalamic Nucleus (STN) for the treatment of the tremor of Parkinson's Disease.

FIG. 5 shows ultrasonic-transducer targeting of the hippocampus and amygdala for the treatment of Tourette's Syndrome.

FIG. 6 shows ultrasonic-transducer targeting of the Hippocampus, Thalamus, Temporal Lobe, and Cerebellum for the treatment of epilepsy.

FIG. 7 shows a block diagram of feedback control for treatment of epilepsy.

### DETAILED DESCRIPTION OF THE INVENTION

It is the purpose of this invention to provide methods and systems for non-invasive deep-brain neuromodulation using ultrasound to treat movement disorders (e.g., motor tremor disorders such as Parkinson's Disease and essential tremor, Tourette's Syndrome, and epilepsy). Also included are the Tourette's vocalizations. Such neuromodulation systems can produce applicable acute or long-term effects. The latter occur through Long-Term Depression (LTD) or Long-Term Potentiation (LTP) via training Included is control of direction of the energy emission, intensity, frequency, pulse duration, firing pattern, and phase/intensity relationships to targeting and accomplishing up-regulation and/or down-regulation.

The stimulation frequency for inhibition is 300 to 500 Hz or lower (depending on condition and patient). In one embodiment, the modulation frequency of lower than approximately 500 Hz is divided into pulses 0.1 to 20 msec. repeated at frequencies of 2 Hz or lower for down regulation. The stimulation frequency for excitation is in the range of 500 Hz to 5 MHz. In one embodiment, the modulation frequency of higher than approximately 500 Hz. is divided into pulses 0.1 to 20 msec. repeated at frequencies higher than 2 Hz for up regulation. The stimulation frequency for excitation (carrier frequency on which the lower-rate stimulation frequency is superimposed) is in the range of 500 Hz to 5 MHz. In this invention, the ultrasound acoustic frequency is in range of 0.3 MHz to 0.8 MHz with power generally applied less than 60 mW/cm² but also at higher target- or patient-specific levels at which no tissue damage is caused. The acoustic frequency is modulated at the lower rate to impact the neuronal structures as desired (e.g., say 300 Hz or lower for inhibition (down-regulation) or 500 Hz or higher for excitation (up-regulation). A wide range in quantity of ultrasound transducers can be used, but typically will fall in the range of one to ten. Ultrasound therapy can be combined with therapy using other devices (e.g., Transcranial Magnetic Stimulation (TMS)). In some embodiments the modulation is pulsed with the pulses up to 2 msec. in length and repeated at 2 Hz or shorter for inhibition and higher for excitation. In other embodiments, mechanical perturbations are applied to the ultrasound transducers to move them radially or axially.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

Transducer array assemblies of this type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon or Blatek in the U.S. are another custom-transducer suppliers. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

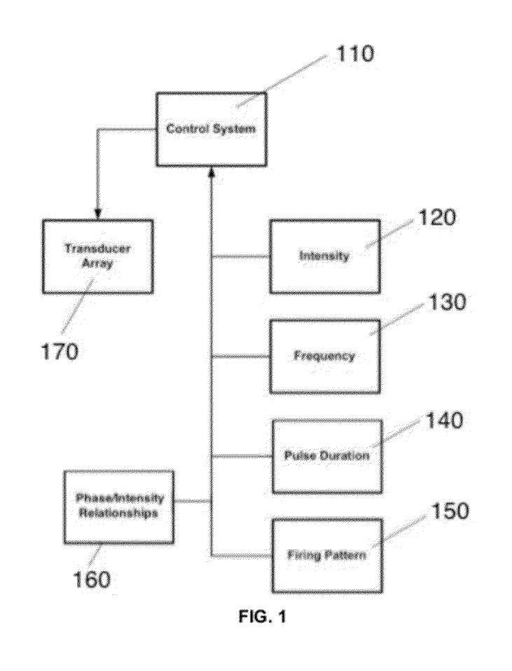

FIG. 1 shows an embodiment of a control circuit for ultrasound neuromodulation variables. The positioning and emission characteristics of transducer array 170 are controlled by control system 110 with control input with neuromodulation characteristics determined by settings of intensity 120, frequency 130, pulse duration 140, firing pattern 150, and phase/intensity relationships 160 for beam steering and focusing on neural targets.

MOTOR TREMOR DISORDERS: In the case of motor disorders, based on experience with Deep-Brain Stimulation (DBS) with implanted electrodes, treatment for Parkinson's Disease or Essential Tremor would typically be 130 pulse per second and Dystonia in the range of 135-185 pulses per second (all superimposed on the carrier frequency of say 0.5 MHz or similar and may be divided into pulses 0.1 to 20 msec. repeated at intervals of 2 Hz or shorter) although this will be both patient and condition specific. In the DBS situation (and ultrasound neuromodulation can be similar), difficult cases are treated with higher rates, up to 250 pulses per second, although it has been found that dystonia will sometimes respond better to lower frequencies, down to 50 pulses per second. Below 50 pulses per second, the tremor can get worse.

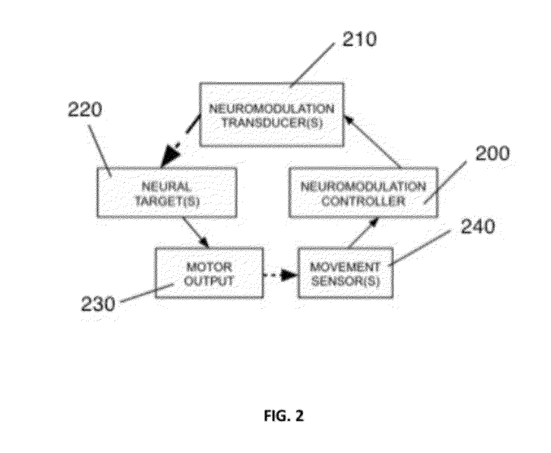

FIG. 2 shows a diagram of a neuromodulation environment that applies feedback to allow real-time control to treat motor disorders. Neuromodulation transducer(s) 210 stimulate neural target(s) 220 that in turn cause motor output 230. Motor output 230 is in turn detected by movement sensor(s) that provide input to the neuromodulation controller that regulates the output of neuromodulation transducer(s) 210. This feedback circuit acts to provide neuromodulation as needed regulate motor output. For example if the magnitude of the motor output increases the system acts to increase the level of neuromodulation causing a decrease in the motor output. The system can be up regulated or down regulated to achieve the desired motor result. This approach causes effective control when the level of tremor varies over time. With respect to such adjustments, having the neuromodulation rate above or below the optimal level can result in motor abnormalities. In another embodiment, the level of neuromodulation is fixed and no feedback loop is present. In still another embodiment, feedback is present but is used for the control of the parameters of DBS.

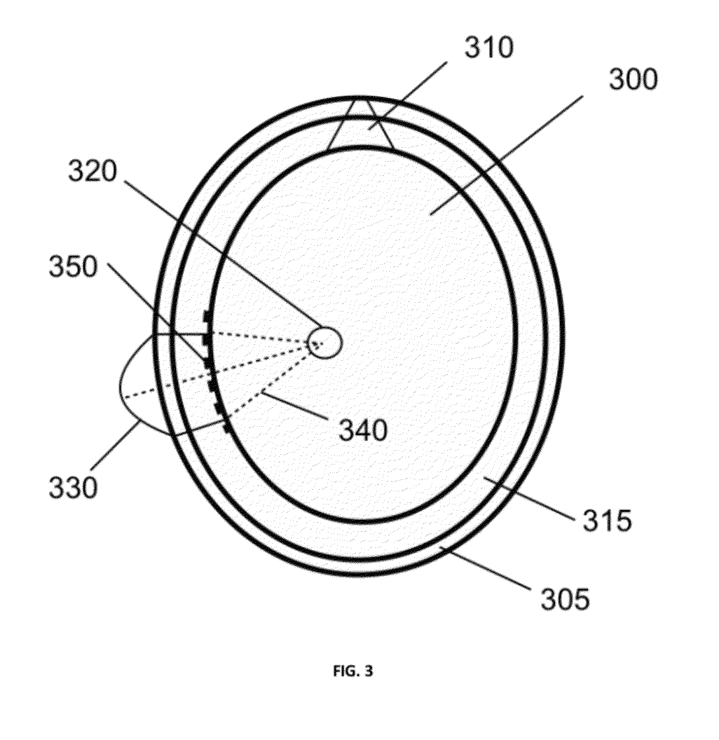

FIG. 3 shows unilateral ultrasonic-transducer targeting of the ventro intermediate nucleus for the treatment of essential tremor. In FIG. 3, patient head 300 with nose 310 showing the orientation. Ventro intermedius nucleus 320 is targeted by ultrasonic transducer with lens 330 mounted on support 315 generating focused ultrasound beam 340. For ultrasound to be effectively transmitted to and through the skull and to brain targets, coupling must be put into place. The ultrasound beam passes through ultrasonic conduction medium 315 (for example, made of Dermasol from California Medical Innovations) with interfaces between the ultrasound transducer and lens 330 on one side and the head 300 on the other with a layer of ultrasound-conduction gel 350.

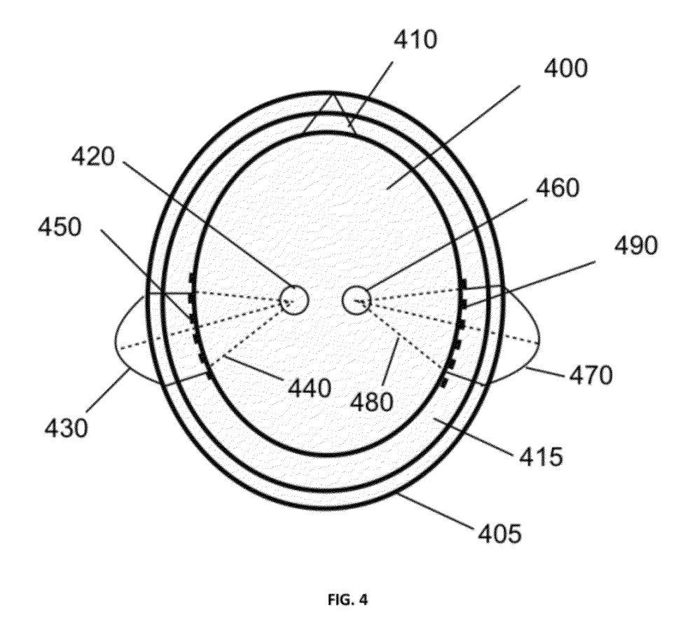

FIG. 4 shows bilateral ultrasonic-transducer targeting of the Subthalamic Nucleus (STN) for the treatment of the tremor of Parkinson's Disease. Patient head 400 with nose 410 showing the orientation contains left STN 420 and right STM 460. Ultrasound transducer and lens 430 targets left STN 420 mounted on support 405 generating ultrasound beam 440. In like manner, ultrasound transducer and lens 470 targets right STN 460 mounted on support 405 generating ultrasound beam 480. As in the case of FIG. 3, ultrasound beams 440 and 480 pass through ultrasound-conduction medium 415 (for example, made of Dermasol from California Medical Innovations) between ultrasound transducers and lens 430 and 470 on one side and head 400 on the other with respectively ultrasound-conduction-gel layers 450 and 490 between ultrasound-conduction medium 415 and head 400. In an alternative embodiment, the same configuration of targets and transducers would be used for the treatment of dystonia. In other embodiments the target for the treatment of Parkinson's Disease or dystonia is the internal globus pallidus (GPi).

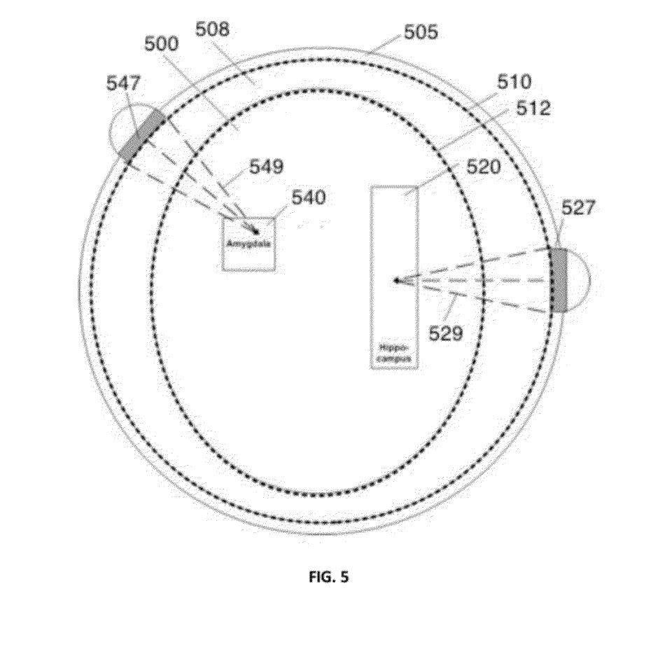

TOURETTE'S SYNDROME: FIG. 5 shows a set of ultrasound transducers targeting to treat Tourette's Syndrome. Head 500 contains two targets, Hippocampus 520, and Amygdala 540. Both are typically down regulated. Note that while these two targets are covered here, one might work as well, or an addition or substitution of other targets (e.g., thalamus, sub-thalamic nuclei, and basal ganglia) identified currently or in the future. These targets are hit by ultrasound from transducers 527 and 547 fixed to track 505. Ultrasound transducer 527 with its beam 529 is shown targeting hippocampus 520, and transducer 547 with its beam 549 is shown targeting amygdala 540. Bilateral stimulation of one of a plurality of these targets is another embodiment. For ultrasound to be effectively transmitted to and through the skull and to brain targets, coupling must be put into place. Ultrasound transmission (for example Dermasol from California Medical Innovations) medium 508 is interposed with one mechanical interface to the frame 505 and ultrasound transducers 527 and 547 (completed by a layer of ultrasound transmission gel layer 510) and the other mechanical interface to the head 500 (completed by a layer of ultrasound transmission gel 512). In another embodiment the ultrasound transmission gel is only placed at the particular places where the ultrasonic beams from the transducers are located rather than around the entire frame and entire head. The invention not only treats motor tics of Tourette's, but the vocalizations as well. In another embodiment, multiple ultrasound transducers whose beams intersect at that target replace an individual ultrasound transducer for that target.

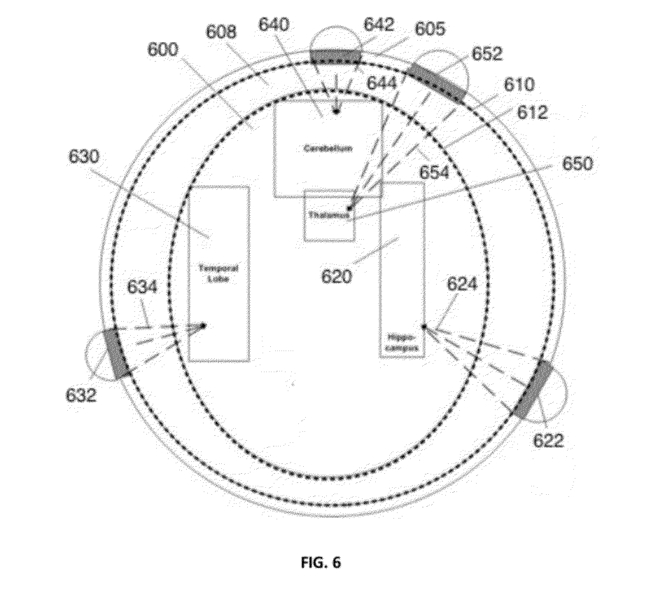

EPILEPSY: FIG. 6 shows a set of ultrasound transducers targeting to treat epilepsy. Head 600 contains four targets, Hippocampus 620, Temporal Lobe 630, the Cerebellum 640, and the Thalamus 650. All of these targets would typically be down regulated although this will be dependent on the relationship among the targets and the particular patient. Note that while these six targets are covered here, others might work as well, or an addition or substitution of other targets (e.g., Amygdala, Dentate Nucleus, and Mamillary Body) identified currently or in the future. The targets shown are hit by ultrasound from transducers 622, 632, 642, and 652 fixed to track 605. Ultrasound transducer 622 with its beam 624 is shown targeting hippocampus 620, transducer 632 with its beam 634 is shown targeting Temporal Lobe 630, transducer 642 with its beam 644 is shown targeting Cerebellum 640, and transducer 652 with its beam 654 is shown targeting Thalamus 650. Bilateral stimulation of one of a plurality of these targets is another embodiment. For ultrasound to be effectively transmitted to and through the skull and to brain targets, coupling must be put into place. Ultrasound transmission (for example Dermasol from California Medical Innovations) medium 608 is interposed with one mechanical interface to the frame 605 and ultrasound transducers 622, 632, 642, and 652 (completed by a layer of ultrasound transmission gel layer 610) and the other mechanical interface to the head 600 (completed by a layer of ultrasound transmission gel 612). In another embodiment the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target.

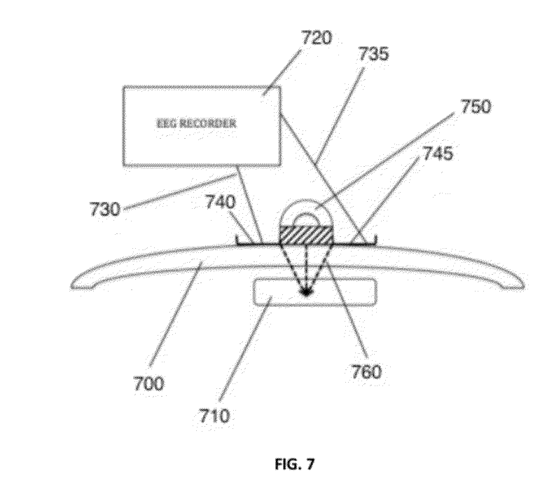

Neuromodulation can be applied continually for frequent seizures of status epilepticus, but another mode of great utility is to use an EEG signal to detect when a seizure is about to occur and have the neuromodulation turned on at the time. As demonstrated by Neuropace (Pless, U.S. Pa. No. 6,466,822, “Multimodal Neural Stimulator and Process of Using It”) applying such a mechanism can prevent a full-blown seizure. This approach uses implanted electrodes, placed during an invasive procedure. Ultrasound neuromodulation has the distinct benefits of being non-invasive, less expensive, and portable so it can be used at home or while working FIG. 7 shows neuromodulation target 710 within patient head 700. Transducer 750 with its beam 760 neuromodulates target 710. A layer of ultrasound conduction gel (not shown) is placed between the face of transducer 750 and head surrounded by skull segment 700. The target can be one of the targets shown in FIG. 6 or others. Multiple transducers can be aimed at multiple targets. Alternatively multiple transducers with beams intersecting at a single target can be used. EEG signals are taken from electrodes 740 and 745 through conductors 730 and 750 respectively to EEG recorder 720. When an incipient seizure is detected in EEG recorder 720, a circuit (not shown) is activated where a trigger is provided to the control unit (not shown) providing neuromodulation output to ultrasound transducer 750 to stop the seizure. EEG signals can also be detected in the ear as taught in a system that included such a detection device combined with a stimulator by Fischell and Upton (US Patent Application Publication US 2003/0195588, “External Ear Canal Interface for the Treatment of Neurological Disorders”).

For any of the cases above, other embodiments have multiple ultrasound transducers with intersecting beams focused on the same target. In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

In still other embodiments, other energy sources are used in combination with or substituted for ultrasound transducers that are selected from the group consisting of Transcranial Magnetic Stimulation (TMS), deep-brain stimulation (DBS), optogenetics application, radiosurgery, Radio-Frequency (RF) therapy, and medications.

The invention allows stimulation adjustments in variables such as, but not limited to, intensity, firing pattern, frequency, pulse duration, firing pattern, phase/intensity relationships, dynamic sweeps, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of deep-brain neuromodulation using ultrasound stimulation, the method comprising:
aiming one or a plurality of ultrasound transducer at one or a plurality of neural targets related to movement disorders, and
applying pulsed power to the ultrasound transducer via a control circuit, whereby movement disorders are alleviated.

2. The method of claim 1, further comprising aiming an ultrasound transducer neuromodulating motor-disorder-related neural targets in a manner selected from the group of up-regulation, down-regulation.

3. The method of claim 1, wherein the effect is chosen from the group consisting of acute, Long-Term Potentiation, and Long-Term Depression.

4. The method of claim 1 where the disease alleviated is motor tremor disorders.

5. The method of claim 4, wherein one or a plurality of targets are selected from the group consisting of subthalamic nucleus, ventralis intermedius of the thalamus and internal globus pallidus.

6. The method of claim 1 where the disease alleviated is Tourette's Syndrome, including both motor tics and Tourette's vocalizations.

7. The method of claim 6 wherein one or a plurality of targets are selected from the group consisting of Hippocampus. Amygdala, Thalamus, Sub-Thalamic Nuclei, and Basal Ganglia.

8. The method of claim 1 where the disease alleviated is epilepsy.

9. The method of claim 8 wherein one or a plurality of targets are selected from the group consisting of Hippocampus, Temporal Lobe, Cerebellum, Thalamus, Amygdala, Dentate Nucleus, and Mamillary Body.

10. The method of claim 1 wherein feedback is applied to trigger ultrasound neuromodulation to stop seizures.

11. The method of claim 1 wherein a single ultrasonic transducer aimed at a given target is replaced by a plurality of ultrasonic transducers whose beams intersect at that target.

12. The method of claim 1, wherein the acoustic ultrasound frequency is in the range of 0.3 MHz to 0.8 MHz.

13. The method of claim 1, where in the power applied is selected from the group consisting of less than 60 mW/cm² and greater than 60 mW/cm² but less than that causing tissue damage.

14. The method of claim 1, wherein a modulation frequency of 50 to 500 Hz is applied for suppression of motor tremor.

15. The method of claim 1 wherein modulation frequency of lower than approximately 500 Hz is divided into pulses 0.1 to 20 msec. repeated at frequencies of 2 Hz or lower for down regulation.

16. The method of claim 1 wherein modulation frequency of approximately 500 Hz or higher is divided into pulses 0.1 to 20 msec. repeated at frequencies higher than 2 Hz for up regulation.

17. The method of claim 1, wherein the focus area of the pulsed ultrasound is 0.5 to 150 mm in diameter.

18. The method of claim 1, wherein mechanical perturbations are applied radially or axially to move the ultrasound transducers.

19. The method of claim 1, wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

20. The method of claim 1, wherein ultrasound therapy is combined with or replaced by one or more therapies selected from the group consisting of Transcranial Magnetic Stimulation (TMS), deep-brain stimulation (DBS), application of optogenetics, radiosurgery, Radio-Frequency (RF) therapy, and medications.
